(19) United States
(12) Patent Application Publication
(10) Pub. No.: US 2021/0393164 A1
(43) Pub. Date: Dec. 23, 2021

(54) JOINT FLEXION INDICATOR DEVICE

(71) Applicant: Juan Cruz TABENA ISERN, Caracas (VE)

(72) Inventor: Juan Cruz TABENA ISERN, Caracas (VE)

(21) Appl. No.: 17/288,770

(22) PCT Filed: Oct. 24, 2019
(86) PCT No.: PCT/IB2019/059154

§ 371 Date: Apr. 26, 2021

(30) Foreign Application Priority Data
Oct. 25, 2018 (CO) NC2018/0011434

(51) Int. Cl. A61B 5/107; A61B 5/11; A61B 5/00

(52) U.S. Cl. CPC A61B 5/1071; A61B 2503/10; A61B 5/458; A61B 5/1121

(57) ABSTRACT

The subject invention corresponds to a joint flexion indicator device connecting a first member to a second member, comprising a first housing connected to the first member; a second housing connected to the second member and a sensor arranged between the housings. The sensor detects an alignment between the housings, wherein the housings are aligned facing each other when the joint is extended. The device of the subject invention allows monitoring the relative position between the members articulated by the joint, which is useful for sports training where certain postures are required to achieve an adequate technical performance. For example, in the case of golf, the device of this invention allows monitoring that an arm and forearm joined by an elbow are aligned, thereby achieving superior performance during the swing stroke. The device also allows monitoring the position and alignment of legs, hands, and feet to correct ergonomic or sports postures.

### FIELD OF INVENTION

The present invention relates to devices for measuring the movement of the entire body or parts thereof, particularly the present invention relates to devices for indicating the flexion of a joint.

### PRIOR ART DESCRIPTION

The ability or physical capacity of an individual is not enough to prove better than others or to excel in a sport. For this reason, athletes train for years not only their physical condition, but also their technique, and it is technique in practicing a sport, which not only improves performance in the sport, but also extends the short career of an athlete. For example, in golf and tennis, two sports that demand a lot from body joints, wrongful practice or movements inflicting joint damage may affect an athlete's career. It is thus necessary to focus on the use of tools to train an athlete, indicating which movements are ideal and which are not.

Accordingly, sensor devices have been used for years to help users during sport training. These devices include sound or visual systems indicating different qualities of the exercise performed by the user.

US 2016/0202755 discloses an array of sensors located superficially on a body joint, in such a way that each sensor extends along said joint.

This arrangement may consist of four to six sensors to measure the movement of a joint, in which each sensor extends along the body joint to be measured, meaning a first sensor extends from the front to the back of the joint.

Structurally, this document discloses that each sensor placed longitudinally to the joint is fastened to a fastening strip at each end of said joint, and in turn, delivers data to a data control unit located on each fastening strip. Such data control unit consists of a processing component; a communication component; a power source; a human-user-computer interface; a user-human-computer interface; and a digital memory.

In one example, the array consists of four sensors extending over the joint and located angularly equidistant from each other, in such a way that each one occupies and measures a quadrant of the joint circumference, respectively. Similarly, this array of sensors may comprise six sensors placed longitudinally to the joint extending over the joint in six different sextants, respectively. In another example, this sensor may also comprise a first accelerometer which is located in front of the body joint and a second accelerometer which is located in the back of the body joint. This array of sensors is located on major joints such as: elbows, shoulders, hips and knees.

Fastening is done with a strip at each end of the sensors, so the need for an additional garment to keep the sensors in position is evident when the body is in motion. Also, due to the number of sensors and their shape, it is necessary to have a computer unit performing signal analysis at all times to determine the type of movement that is being performed. This proves inefficient and forces the device to be more expensive in terms of energy, and also costs more work to use it, given a garment keeping sensors in position is necessary.

On the other hand, CN1657325A discloses a system for monitoring important components of body movement and flexion during kinetic activities, and providing real-time biofeedback to the user in an intuitive and audible manner.

The CN1657325A system comprises a trident-shaped base having one or more flexion sensors fitting into a thin pocket at the end of the trident-shaped base where the sensor connection extends from the end of the trident and extends beyond the length of the trident-shaped base and connects to the housing of a system module, where the trident-shaped base fits conveniently into an elbow orthopedic appliance. The system module housing is thus attached to the orthopedic appliance with Velcro or similar fastening means, and wherein the system module housing holds a tone generator, which is setup to step-by-step increase the tone with respect to the measurement taken by the flexion sensor.

In particular, said prior art shows a device comprising a sensor placed at the end of a trident structure allowing the sensor to be accommodated to measure elbow flexion (for example), and the module emits monotones that change tone to indicate a change in the flexion angle of the joint or point of interest, the feedback thus being instantaneous. Among the points of interest, elbows, hip and knee of a golf player can be noted.

Given the shape of the sensor, it is possible to install it in different elements, such as elbow pads, kneepads, among others. However, as they are not an integral part of them, it is necessary to use additional elements for installation, and even so, it cannot be guaranteed they will not move erroneously or fall off during joint movement. Also, due to the shape of sensors that may be used, it is necessary to have a signal processing stage that can identify differences between the flexion angles. Shape also requires an additional step when placing the device in a bracelet, and because it takes up space, it may become uncomfortable when practicing a sport, given knee or elbow pads are usually designed to leave little space to adjust to any exercise and to the shape of the joint.

Finally, US20140194781A1 shows a portable device with feedback features, which comprises: a sleeve, preferably tube-shaped open to extend over a joint; at least one sensor embedded in or within the sleeve and configured to detect at least one leg angle; a control package embedded to the sleeve; at least one sensor embedded in or within the sleeve and configured to detect at least one leg angle; a feedback element, which can indicate using a light-emitting diode an unsafe orientation of the joint, and placed in or within a control package, and having at least a first and second configuration; where the processor is also connected to a battery.

Specifically, the feedback provided by the device in this prior art document is that when the joint moves to an unsafe or dangerous position, it warns the user by means of sound, light or assists providing substantially rigid structural support to the joint. These mechanisms are controlled by a control package or monitoring in conjunction with the joint sleeve.

This invention, like the others, requires a signal processing stage, which demands additional energy consumption.

Considering the above, the need for a sensor to be placed comfortably, and which does not consume much energy, not only to keep the weight of the device to a minimum, but also to increase the time of use in order to increase activity time, is clear.

### BRIEF DESCRIPTION OF THE INVENTION

The present invention relates to a joint flexion indicator device, connecting a first member to a second member, comprising a first housing connected to the first member; a second housing connected to the second member and a sensor arranged between the housings. The sensor detects an alignment between the housings, wherein the housings line up facing each other when the joint is extended and thus form an opening between them.

The device of the present invention allows monitoring the relative position between the members connected by a joint, which is useful for sports training where certain postures are required to achieve appropriate technical performance. For example, in the case of golf, the device of this invention allows monitoring that an arm and forearm joined by an elbow are aligned, thereby achieving superior performance during the swing stroke. The device also allows monitoring the position and alignment of legs, hands, and feet to correct ergonomic or sport postures.

### BRIEF DESCRIPTION OF THE FIGURES

FIG. 1 corresponds to an isometric view of a device embodiment of the present invention, including a detail of an adjustment screw connected to a sensor.

FIG. 2 corresponds to an exploding view of the device in FIG. 1.

FIG. 3 corresponds to an isometric view of a device embodiment of the present invention comprising a sleeve, where the device connects to an arm and forearm that are extended and aligned.

FIG. 4 corresponds to an isometric view of the device in FIG. 3, where the arm and forearm are flexed and misaligned.

FIG. 5 corresponds to an isometric view of a device embodiment of the present invention comprising belts.

### DETAILED DESCRIPTION OF THE INVENTION

In general, the joints that wear the most during training for any sport, or during physical labor, are the elbows and knees. These joints connect two limbs of the arm or leg, and in addition, this connection allows the movement of one limb with respect to the other, where such movement can be flexion or rotation. Also, joints such as wrists, shoulders, and ankles may be damaged by ergonomically appropriate postures during sports or work.

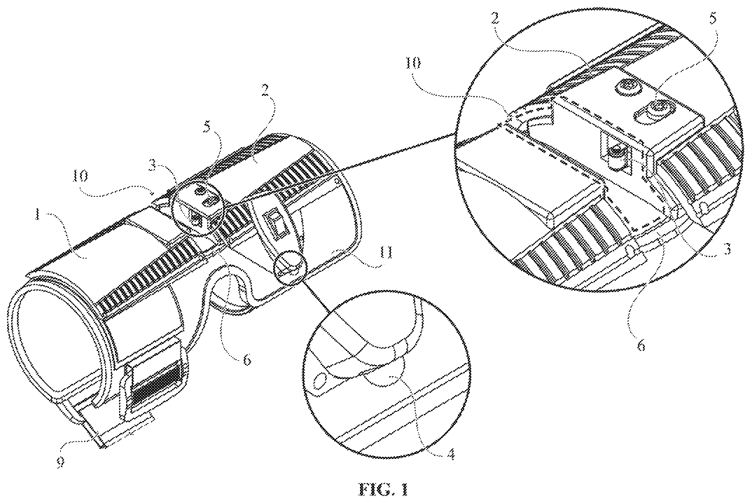

Referring to FIG. 1, the present invention corresponds to a joint flexion indicator device connecting a first member to a second member, comprising a first housing (1) connected to the first member; a second housing (2) connected to the second member; a sensor (3) arranged between the first housing (1) and the second housing (2), where the sensor (3) detects an alignment of the first housing (1) with the second housing (2); and where the first housing (1) and the second housing (2) align opposite each other when the joint is extended and thus forming an opening (10) between them.

In the present invention, the first and second limbs shall be understood as parts of the human body, or parts of an animal, which are articulated by a joint. For example, the first limb and second limb are selected from fingers, phalanges, hands, forearms, arms, thighs, feet, and calves. Also, the first extremity may be part of a limb such as the arm or leg, while the second limb may be part of the trunk.

The first housing (1) adjusts to the first member, while the second housing (2) fits the second member. These housings (1, 2) are adjusted to the members so they do not slip, or move from away from those members. This is in order to prevent the sensor (3) from being activated in erroneous positions generating a mistaken alert.

Preferably, the first housing (1) and the second housing (2) are channel-shaped, so each housing (1, 2) may partially wrap a limb or extremity, allowing it to connect to the limb safely and ergonomically. The housings (1, 2) are aligned in front of each other when the joint connecting the two limbs is extended.

Optionally, the housings (1, 2) have a cushioned element on the inside of the channel shape providing comfort to a user and preventing injuries or lacerations caused by the housings (1, 2). For example, the padded element is selected from the group that includes polymeric foams, non-woven textiles, microfiber textiles, woven textiles with filler, textiles with silicone fibers, elastomers (e.g. rubber, elastane, spandex fibers), rayon, woven fabrics with polymeric fibers (e.g., polyester, polypropylene, polyester-viscose, polyamide 6, polyamide 6.6, polyamide), animal fiber fabrics (e.g. wool fibers, silk, vicuña, cashmere, alpaca, or rabbit fur), vegetable fibers (e.g. cotton, flax, cellulose), silicone veils and stockings or combinations thereof.

Preferably, the housings (1,2) are made of a light and resistant material, but at the same time it has an adequate formability to adapt to the geometry of the user's members. For example, polymers (polyester resins, vinylester, epoxy, vinyl) reinforced with fibers (e.g., glass, aramid, polyester), allow the housings (1,2) to be manufactured in molds by manufacturing processes such as spraying, hand lay-up, resin infusion processes, resin transfer molding (RTM), Reaction Injection Molding (RIM). Vacuum-Assisted Resin Transfer Molding (VARTM), thermoforming, pultrusion, and combinations thereof.

On the other hand, thermoplastic materials allow the housings (1,2) to be manufactured by injection, rotational molding, three-dimensional printing or thermoforming; such thermoplastic materials may be polyethylene (PE), high density polyethylene (PEHD), ultra-high density polyethylene (PEUHD), polyvinyl chloride (PVC); polychlorinated vinyl chloride (CPVC); polyethylene terephthalate (PET), polyamides (PA) (for example, PA12, PA6, PA66); polychlorotrifluoroethylene (PCTFE); polyvinylidene fluoride (PVDF); polytetrafluoride ethylene (PTFE); ethylene-chlorotrifluoroethylene (ECTFE).

Preferably, the housings (1,2) are manufactured from a biocompatible material, which complies with ISO-10993: “Biological Evaluation of Medical Devices” Some materials are: Polyaryletherketone (PAEK), polyetheretherketone (PEEK), high-density polyethylene (HDPE), ultra-high molecular weight polyethylene (UHMWPE), polymethylmethacrylate (PMMA), polysulfones (PSU), polyetherimide (PEI), polyphenylsulfone (PPSU) and polyphenylene sulfide (PPS), Commercially Pure Titanium (ASTM F67), titanium alloys ASTM B265 (standard specification of titanium and titanium in strip, sheet and plate form) and AISI 316L Stainless Steel, Acrylonitile butadiene styrene (ABS), polycarbonate, polyamide, polyester, polyvinyl chloride (PVC).

On the other hand, in order to secure the housings (1, 2) to the members, fasteners, sleeves, meshes, bands, belts, chains, straps, textiles, and combinations thereof can be used.

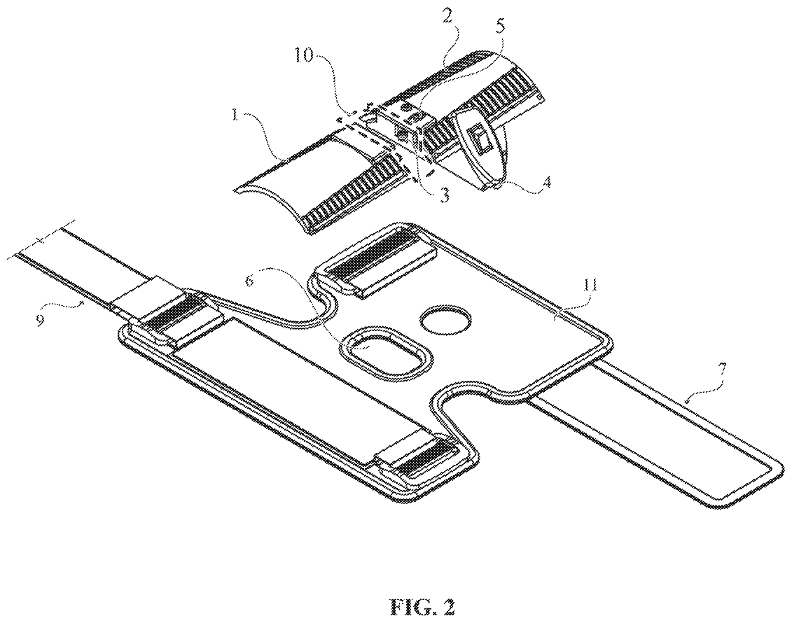

For example, referring to FIG. 1 and FIG. 2, the housings (1,2) are secured to the members by means of a flexible panel (11) which has belts (7, 9). This flexible panel (11) has a first belt (9) and a second belt (7), where the first belt (9) is connected to the first housing (1) and the second belt (7) is connected to the second housing (2), and where the flexible panel (11) keeps the first housing (1) and the second housing (2) connected to the first member and the second member by means of the belts (7, 9).

The flexible panel (11) functions as the body of the device and allows the device to be adjusted to the desired joint. Preferably, the flexible panel (11) comprises a space (6) in which the external vertex of articulation is positioned, for example, in the case where the device is used on an arm connecting the housings (1, 2) to the arm and forearm, the space (6) is located on the elbow, whereas when the device is used on one leg, where the housings (1, 2) are attached to the thigh and calf, the space (6) is then located in the knee position.

Preferably, the flexible panel (11) is made of a material selected from the group comprising polymeric foams, non-woven textiles, microfiber textiles, woven textiles with filler, textiles with silicone fibers, elastomers (e.g. rubber, elastane, spandex fibers, rayon, fabrics with polymeric fibers (e.g. polyester, polypropylene, polyester-viscose, polyamide 6, polyamide 6.6, textured polyamide), animal fiber fabrics (e.g. wool fibers, silk, vicuna, cashmere, alpaca, and rabbit fur), vegetable fibers (e.g. cotton, linen, cellulose), silicone or combinations thereof.

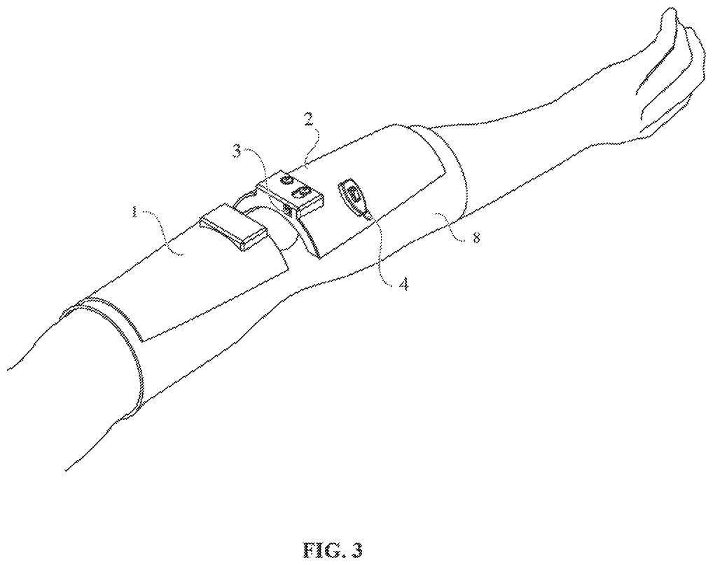

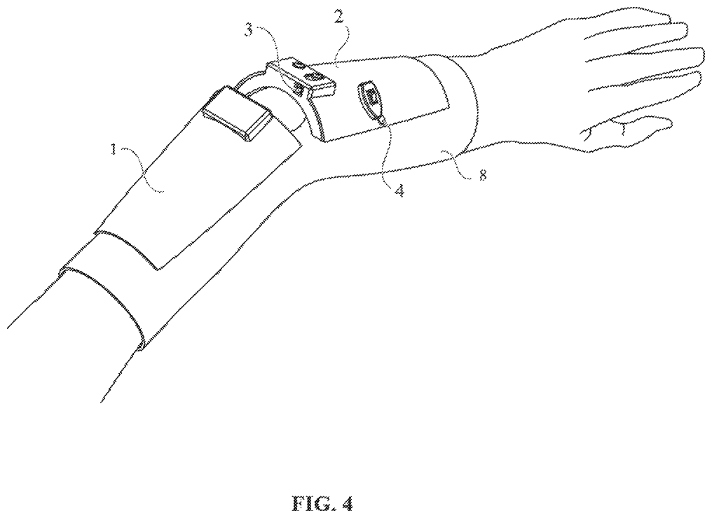

On the other hand, referring to FIG. 3, and FIG. 4, the device of the present invention may comprise a sleeve (8) surrounding the members to which the housings are connected (1, 2). In this case, these housings (1, 2) are connected to the sleeve (9) with means of an attachment selected from the group which includes woven joints, strips, staples, straps, chains, screws, rivets, bolts, equivalent joints known to a person moderately skilled in the art and combinations thereof. In particular, if the means of attachment are bolts, screws, or rivets, it is preferable that these be rounded tipped, or that they are inside the housings (1,2) in countersunk or deep holes, which prevents them from hurting the user during the use of the device.

Preferably, the sleeve (8) is a sleeve made of a material selected from the group that includes polymeric foams (e g, polyurethane foams, polyethylene, polyester, ethylene vinyl acetate (EVA), non-woven textiles, microfiber textiles, woven textiles with filler, textiles with silicone fibers, elastomers (e.g., rubber, elastane, spandex fibers), rayon, woven polymeric fibers (e g, polyester, polypropylene, polyester-viscose, polyamide 6, polyamide 6.6, textured polyamide), animal fiber fabrics (e.g. wool fibers, silk, vicuna, cashmere, alpaca, or rabbit fur), vegetable fibers (e.g. cotton, linen, cellulose), silicone veils and stockings or combinations thereof.

For example, the sleeve (8) may be a woven sleeve made of yarns consisting of filaments and/or spandex in combination with rigid filaments and/or fibers, e.g. polyester filaments and/or fibers or textured polyamide, cotton filaments and/or fibers, or bi-component synthetic fibers.

Preferably, the sleeve (8) comprises a space (6) for a joint to rest, a proximal part wrapped around the first limb, a distal part wrapped around the second limb. In this case, the first housing (1) is assembled to the proximal part, while the second housing (2) is assembled to the distal part.

On the other hand, preferably, the opening (10) located between the first housing and the second housing coincides with the space (6) of the sleeve for the joint to rest, where the opening (10) has a distance ranging between 5 mm and 25 mm. Accordingly, if the device is used on an arm connecting the housings (1, 2) to the arm and forearm, the space (6) is located at the elbow and the opening (10) is located above the space (6). In this way, if the elbow flexes, the opening (10) opens, which is detected by the sensor (3).

For example, in the event the device is used for sports training, in the case of golf, in order to execute a proper swing the user must keep his/her arm straight, aligning the arm and forearm. Therefore, the device of the present invention is used in such a way that the second housing (2) is installed on the forearm while the first housing (1) is installed on the arm, so when the elbow is flexed, the sensor (3) detects such flexion.

Optionally, the opening (10) located between the first housing and the second housing is vertically opposite with the space (6) of the sleeve for the joint to rest, where the opening (10) has a distance ranging between 5 mm and 25 mm.

On the other hand, the device of the present invention may include an indicator (4) connected to the sensor (3), where the indicator (4) indicates the flexion of a joint connecting a first limb with a second limb. The indicator (4) may be located in one of the housings (1, 2), or it may be located outside of the housings.

Referring to FIG. 3, the housing (1) and housing (2) are shown aligned when a user's arm is extended. In this arm position, sensor (3) is deactivated.

Referring to FIG. 4, it is shown housing (1) and housing (2) are not aligned, when a user's arm is flexed. In this arm position, the sensor (3) is activated when misalignment of the housings (1, 2) is detected.

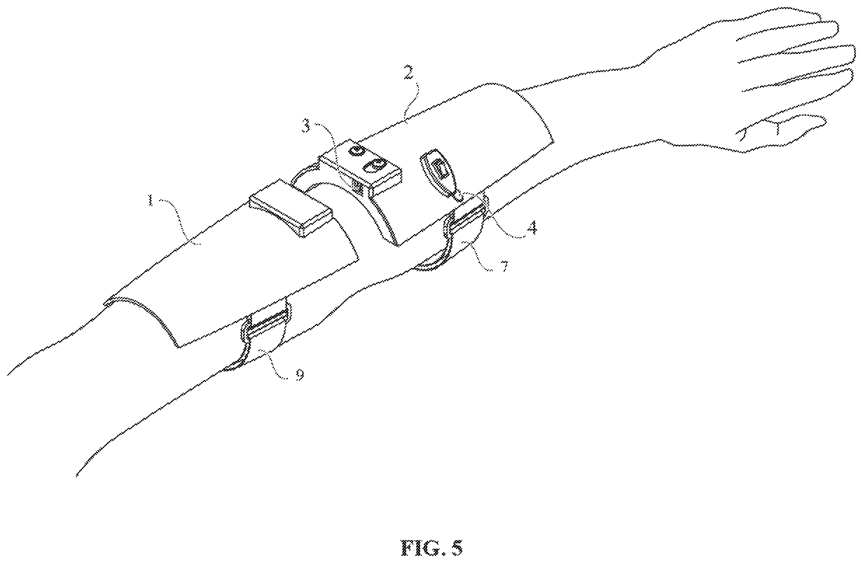

Referring to FIG. 5, in another embodiment, the first housing (1) includes a support belt (7) allowing it to be connected to a joint member, the support belt (7) may be adjusted in such a way it adapts to different user sizes. The second housing (2) is also fastened with a belt to the other member.

Preferably, the indicator (4) and sensor (3) are electronic devices connected to a battery. For example, the battery may be selected from a group including alkaline batteries, nickel batteries, or from a group of rechargeable lithium-ion batteries, e.g. LFP batteries, NMC batteries. Li—S batteries, LiPo batteries and other equivalent batteries known to an averaged skilled person in the art, lead-acid batteries, advanced lead acid batteries, NiMH metal hydride batteries, nickel cadmium (NiCd) batteries, zinc bromide batteries, sodium nickel/chlorine NaNiCl batteries, zinc air batteries, redox vanadium batteries, other equivalent batteries known to an averaged skilled person in the art, and combinations thereof.

In addition, the indicator (4) may be selected from a group that includes light emitting diodes, light emitting diodes, liquid crystal displays, LED displays, light bulbs, sound emitting indicators, bells, baffles, loudspeakers, resonance boxes, vibration generators, oscillating motors, vibration motor, a resonant linear actuator, and combinations thereof.

Similarly, the sensor (3) may be selected from a group consisting of piezoelectric sensors, actuators, switches, end-of-line sensors, fiber optic sensors, ultrasonic sensors, laser sensors, vibration generators, oscillating motors, vibration motor, resonant linear actuators, and combinations thereof.

Optionally, the indicator (4) may be placed on any part of the user's body, outside the joint flexion indicator device.

On the other hand, the device of the present invention may comprise a control unit connected to the sensor (3) using a wireless communication unit; and an indicator (4) connected wirelessly to the control unit. The control unit collects signals from the sensor (3) that are generated when the members connected to the housings (1, 2) are in inadequate positions.

It will be understood that inappropriate positions depend on the type of action or posture sought, for example, that the members are aligned, or that they form a specific angle to each other.

### Example 1

An embodiment of the device in the present invention was designed and manufactured comprising a sleeve (8) with a space (6) for a joint to rest, a proximal part surrounding the first limb, a distal part surrounding the second limb, where the sleeve (6) is made of an elastic fiber that fits around the joint. In addition, the device comprises a first housing (1) attached to the proximal part, a second housing (2) attached to the distal part. In this case the housings (1, 2) are made of polyamide 6.6. In addition, the sensor (3) is a limit switch sensor (3) arranged between the first housing (1) and the second housing (2) that detects the alignment of the first housing (1) with the second housing (2), where the first housing (1) and the second housing (2) align with each other when the joint is fully extended.

### Example 2

A similar device to example 1 was designed and manufactured, which also includes an adjustment screw (5) assembled to the limit switch sensor (3) so the device sensitivity may be adjusted mechanically.

### Example 3

A similar device to example 2 was designed and manufactured, which also includes a battery connected to the limit switch sensor (3), and an indicator (4) connected to the limit switch sensor (3) and to the battery, where the indicator (4) and the sensor (3) are connected to a switch that turns the device on and off.

### Example 4

A similar device to example 3 was designed and manufactured, where the opening (10) between the first housing (1) and the second housing (2) coincides with the space (6) of the sleeve (8) for the joint to rest.

### Example 5

A similar device to example 3 was designed and manufactured, where the indicator (4) connected to the limit switch sensor (3) is located in the second housing (2) in such a way the user can see it at all times.

### Example 6

A similar device to example 5 was designed and manufactured, which also has a control unit connected to the limit switch sensor (3) with a wireless communication unit; an indicator (4) connected wirelessly to the control unit.

### Example 7

Referring to FIG. 1 and FIG. 2, an embodiment of the device in the subject invention comprises a first housing (1) connected to the first member; a second housing (2) connected to the second member, a sensor (3) arranged between the first housing (1) and the second housing (2) that detects the alignment of the first housing (1) with the second housing (2), an indicator (4) connected to the sensor (3) and a flexible panel (11) that comprises a first belt (9) and a second belt (7) to fit with the first belt (9) to the first member and with the second belt (7) to the second member, where the flexible panel (11) keeps the first housing (1) and the second housing (2) connected with the first member and the second member; and where the first housing (1) and the second housing (2) line up opposite each other when the joint is fully extended.

It must be understood that the present invention is not limited to the embodiments described and illustrated herein, and the averaged skilled person in the art will understand that numerous variations and modifications can be made that do not depart from the spirit of the invention, which is only defined by the following claims.

1. A joint flexion indicator device connecting a first limb with a second limb, which comprises:
a. a first housing (1) connected to the first member;
b. a second housing (2) connected to the second member;
c. a sensor (3) arranged between the first housing (1) and the second housing (2), where the sensor (3) detects an alignment of the first housing (1) with the second housing (2); and
where the first housing (1) and the second housing (2) line up facing each other when the joint is extended and form an opening (10) between them.

2. The device of claim 1, where the housing (1) and housing (2) comprise a channel shape, where the channel shape of each housing (1, 2) partially surrounds a limb.

3. The device of claim 1, where the first housing (1) includes a first belt (9) that secures the first housing (1) to the first member, and where the second housing (2) includes a second belt (7) that secures the second housing (2) to the second member.

4. The device of claim 1, which also comprises a flexible panel (11) having a first belt (9) and a second belt (7), where the first belt (9) is connected to the first housing (1) and the second belt (7) is connected to the second housing (2), and wherein the flexible panel keeps the first housing (1) and the second housing (2) connected to the first limb and the second limb by means of the belts (7, 9).

5. The device of claim 1, which also comprises a sleeve (8) that has a space (6) for a joint to rest, a proximal part that surrounds the first limb, and a distal part that surrounds the second limb.

6. The device of claim 1, characterized because the sensor (3) is a limit switch sensor.

7. The device of claim 6, which also comprises an adjustment screw (5) assembled at the base of the sensor (3), where the adjustment screw (5) moves the sensor (3) to adjust its sensitivity mechanically.

8. The device of claim 5, where the opening (10) located between the first housing (1) and the second housing (2) is vertically aligned with the space (6) of the sleeve (8).

9. The device of claim 1, where the opening (10) located between the first housing (1) and the second housing (2) forms a separation between the housings (1, 2) between 5 mm and 25 mm.

10. The device of claim 1, which also includes an indicator (4) connected to the sensor (3).

11. The device of claim 10 where the indicator (4) is selected from a group that includes light emission indicators, light-emitting diodes, liquid crystal displays, led displays, light bulbs, sound emission indicators, buzzers, loudspeakers, resonance boxes, vibration generators, oscillating motors, vibration motor, a resonant linear actuator, and combinations thereof.

12. The device of claim 10 where the indicator (4) is located outside the housings (1, 2).

13. The device of claim 10 where the indicator (4) is located in the second housing (2).

14. The device of claim 1, where the sensor (3) is selected from a group comprised of piezoelectric sensors, actuators, switches, end-of-line sensors, fiber optic sensors, ultrasonic sensors, laser sensors, vibration generators, oscillating motors, vibration motor, resonant linear actuators, and combinations thereof.

15. The device of claim 10, which also comprises a control unit connected to the sensor (3) with a wireless communication unit, and an indicator (4) connected wirelessly to the control unit.
